(19) United States
(12) Patent Application Publication
(10) Pub. No.: US 2006/0223988 A1
(43) Pub. Date: Oct. 5, 2006

(54) High Resolution Methods and Precipitating Reagents for Isolating Proteins from Proteinaceous Material

(75) Inventor: Gerald L. Maurer, Cincinnati, OH (US)

Correspondence Address: LAFKAS PATENT LLC, 7811 LAUREL AVENUE, CINCINNATI, OH, 45243, US

(73) Assignee: NATIONAL RESEARCH LABORATORIES, LTD., Cincinnati, OH (US)

(21) Appl. No.: 10/907,531

(22) Filed: Apr. 5, 2005

(51) Int. Cl. C07K 1/30

(52) U.S. Cl. 530/412

(57) ABSTRACT

Novel high resolution methods and precipitating reagents for isolating desired protein populations from proteinaceous materials are disclosed. Uniquely, the methods and precipitating reagents of this invention are suitable for selectively and sequentially isolating consistently in high yields desired protein populations from various proteinaceous materials based upon the physical and chemical characteristics inherent in, and/or peculiar to, the desired protein populations. In addition, desired protein populations isolated with the new and vastly improved methods and precipitating reagents of this invention are highly concentrated and substantially pure and biologically active. The precipitating reagents basically are an alkali metal salt of a polyfunctional organic acid, exemplified by tri-potassium citrate monohydrate or tri-sodium citrate dihydrate, and a buffer for adjusting pH. Sources of proteinaceous materials may include, for example, animal or vegetable fluids and extracts.

### BACKGROUND

Blood is a unique tissue composed of two basic fractions which include plasma and specialized cells. Plasma is the liquid fraction and represents a highly complex mixture of molecular entities both in solution and in suspension which range from small molecules, such as hydrogen ions, to large proteins having molecular weights of several million daltons. The proteins present within the plasma fraction have been presently characterized under seven general categories: (1) albumin, (2) alpha₁ globulins, (3) alpha₂ globulins, (4) beta globulins, (5) fibrinogen, (6) gamma globulins, and (7) prealbumin. With respect to the fractionation of these plasma proteins, the methods generally recognized as being most successful are based upon the use of cold ethanol under precise conditions of pH, temperature, ionic strength and protein concentration. These are disclosed in, inter alia, U.S. Pat. No. 2,390,074 to Cohn and U.S. Pat. No. 2,469,193 to Cohn. Other methods such as those utilizing aqueous solutions of citrate have been utilized for fractionating proteins from plasma as described in U.S. Pat. No. 2,867,567 to Bidwell. However, when such citrate solutions are added to human or horse plasma, the products generated therefrom unfortunately show little or no activity. Each year millions of liters of “whole plasma” are obtained for use in the preparation of a variety of therapeutic substances. Notwithstanding the fact that “whole plasma” has often been used it is well established that the utilization of the individual proteins within the plasma is far superior to using “whole plasma”, and this has become generally accepted in the medical community. The specialized properties of the plasma proteins render them extremely important in treating specific disease states in humans. For example, the plasma proteins have been heretofore utilized to treat shock due to circulatory volume deficiencies, to treat specific pathogens such as tetanus, to prevent measles, hepatitis, etc., and for volume expansion. Further, some of the plasma proteins, such as Factor VIII, a beta globulin, are intimately involved in the all important coagulation process of the blood and ultimately in its conservation.

Since the total protein content of a normal human being represents only about 6% of the total plasma volume, i.e., approximately 6 grams per 100 milliliters of plasma, it is essential that the procedures utilized for fractionating these specific proteins from the plasma fraction have the ability to generate a highly concentrated yet purified preparation with respect to a desired protein constituent without adversely affecting its biological activity. Unfortunately, the techniques heretofore available for isolating individual or selected proteins from the plasma fraction, as well as other proteinaceous materials, are generally inefficient and unrefined. For instance, the therapeutic application of gamma globulin preparations are limited by virtue of the denaturation of the globulin molecules, rendering them “aggregated” and immunogenic in the recipient when administered intravenously. Further, intramuscular injections of optimal therapeutic amounts of normal gamma globulins are generally unsatisfactory due to the large amounts of gamma preparations needed to establish in vivo levels. Additionally, alteration and/or inactivation of specific gamma globulin functions are generally severe in the fractionation methods currently available. For example, under present methods, of a possible 100 grams of gamma globulin containing 100 units of specific activity in the normal plasma pool, fractionation yields only from about 30 to about 70 grams of protein having, perhaps, only from about 2 to about 10 units of activity.

The first practical large-volume protein fractionation methodology was developed by Cohn, as aforesaid, and involved the precipitation of proteins by altering their solubilities utilizing ethanol, salts, temperature and pH control. The Cohn methods, with some modification, are the only procedures heretofore acceptable by the United States Food and Drug Administration for the preparation of, for instance, gamma globulins, albumin, and fibrinogen. These protein fractions, however, are the only plasma fractions having utility currently, yielding only from about 50% to about 70% of the theoretical protein values. Factor VIII, on the other hand, is obtained by several other methods including cryoprecipitation, affinity chromatography, glycine precipitation, etc. At the present, however, Factor VIII yields are only between about 30% and about 50% of the theoretical protein values. The low yields are generally due to the harshness of the precipitating reagents and conditions associated with the methodologies employed which causes alteration of structure and, therefore, of function.

In Europe and most countries other than the U.S.A., the ammonium sulphate “salt-precipitation” method is used since gamma globulin of higher purity in contrast to the Cohn methods is possible, although the yields are very low by comparison for the same reasons as recited above.

Dairy products, such a bovine milk, also represent excellent sources of proteinaceous materials which can be fractionated. For example, the proteins present in bovine milk are divided into three general categories, that include casein, lactoalbumin and lactalbumin and are present in concentrations of about 3% w/v. Presently, only casein, which constitutes the major protein component in dairy products such as cheese and yogurt, is being utilized effectively. The balance of the milk proteins generally remain in the resulting fluid, known as why, which results from the manufacture of cheese and yogurt. Whey represents the major by-product of the dairy industry and is disposed of by methods, such as reverse osmosis-ultrafiltration and spray drying. Unfortunately, spray drying is the more common method utilized which generally results in the denaturation and loss of use of the balance of the milk proteins due to the heat and mechanical shearing associated with the drying process.

In summary, previous attempts or approaches have been taken to fractionate out protein entities from various proteinaceous materials such as plasma fractions. Heretofore, no satisfactory method has been developed which can overcome the problems aforementioned, i.e., concentration, purification, and biological activity. In particular, the known methods generally rely upon a precipitating reagent along with a plurality of conditions which are burdensome, harsh and time-consuming, as well as being physiologically insensitive. In fact, as demonstrated above, most of the known methods are extremely inefficient due to the harshness of the reagents employed. Moreover, the prior methods have not been sensitive enough to isolate protein entities that are present in small amounts in the plasma fraction.

In other words, all of the methods for fractionating proteins provided hitherto are generally of low resolution quality and, therefore, invariably necessarily lack the ability to isolate in high yields highly concentrated protein entity preparations having both a high level of purity and biological activity from proteinaceous materials, such as plasma. Current technology is simply unable to provide satisfactory products to effect the realization of the potentials needed for various protein fractions, such as the plasma proteins. Consequently, there are strong medical and commercial needs for a high-resolution fractionation method that can isolate in high yields highly concentrated and purified protein entity preparations from proteinaceous material effectively without adversely affecting their biological activity.

### SUMMARY

In brief, the present invention seeks to alleviate the above-mentioned problems and shortcomings of the present state of the art through the discovery of novel, high resolution methods and precipitating reagents for isolating desired protein populations from proteinaceous materials. The specific protein populations isolated are highly concentrated and substantially pure and substantially biologically active. In a preferred embodiment, the present invention is directed to a high resolution method of isolating a desired protein population from a proteinaceous material comprising providing a critical concentration of a precipitating reagent comprised of an alkali metal salt of a polyfunctional organic acid which corresponds to the desired protein population to be isolated, adding the precipitating reagent at a concentration less than about the critical concentration to the proteinaceous material for isolating therefrom an undesired protein population, and adding the precipitating reagent at essentially the critical concentration to the fractionated proteinaceous material to further isolate therefrom the desired protein population where in the desired protein population is concentrated substantially pure and biologically active. The protein population may be isolated from various types of proteinaceous materials such as bacterial, animal or vegetable fluids and extracts. Such animal fluids include, but are not limited to, blood, plasma, urine, milk, and amniotic, placental, or fetal fluid. Bacterial, animal and plant extracts may include such things as culture fluids, liver extract, animal feces, corn and soybean extracts, and the like.

In another exemplary embodiment, the present invention is directed to precipitating from a gross protein fraction solubilized in an aqueous liquid undesired protein populations to isolate in the aqueous liquid in solubilized form a specific or desired protein population wherein the term “solubilized” includes colloidal and particulate suspensions. Therefore, this modified approach comprises adding the precipitating reagent at essentially the critical concentration to a proteinaceous material to precipitate therefrom a gross protein population containing the desired protein population, resolubilizing the gross protein population in an aqueous liquid, and adding the precipitating reagent at a concentration less than about the critical concentration to the resolubilized gross protein population for substantially isolating in the liquid in soluble form substantially isolating in the liquid in soluble form the desired protein population.

In another exemplary embodiment, the present invention is directed to high resolution precipitating reagents for selectively isolating a desired protein population from a proteinaceous material comprising an alkali metal salt of a polyfunctional organic acid in a concentration suitable for selectively isolating the desired protein population, and a buffer for adjusting pH of the reagent whereby the desired protein population is selectively isolated from the proteinaceous material in a substantially pure and biologically active form. The alkali metal salts of polyfunctional organic acids are preferably derived from monovalent alkali metals of citric acid and tartaric acid, such as lithium citrate, potassium citrate, and sodium citrate. In particular, tri-potassium citrate monohydrate and tri-sodium citrate dihydrate are especially suitable for formulating precipitating reagents for use with the methods of this invention. In addition, the buffers incorporated into the precipitating reagent include, for instance citric acid or hydrochloric acid, for adjusting pH of the reagent that may range, for example, from about 4 to about 9. When the methods and precipitating reagents of this invention are utilized for selecting specific or desired human protein populations, however, the pH of the precipitating reagent should be from about 7 to about 7.55, and preferably from about 7.35 to about 7.45, the “physiological” pH range, to avoid denaturation and decomposition of the protein populations being isolated.

Therefore, the new and vastly improved methods and precipitating reagents of this invention for isolating proteins provide novel means for selectively and sequentially removing from a colloidal suspension or solution selected protein populations in high yields that are substantially pure and biologically active based on certain physical characteristics inherent in, and peculiar to, the selected protein populations. Remarkably, the nature of the precipitating reagents employed is such that extremely high resolution of specific protein populations of a complex mixture is possible by selectively rendering them insoluble with reference to the continuous phase of the aqueous system. This selective insolubility makes the high resolution possible with the methods and reagents of this invention. During their insolubility, the desired or specific protein populations so insolubilized may be removed from the aqueous medium via conventional liquid-solid separating procedures. The desired or specific protein populations, so isolated in an insoluble form, can be easily resolubilized by simple addition of water, normal saline or mixtures thereof.

In another exemplary feature of the present invention, it is directed to inherent advantages in connection with the isolation and concentration techniques of proteinaceous materials. For instance, plasma contains, in addition to desirable components, some definitely undesirable components with reference to, for example, pharmaceutical manufacturing. By proper adjustment of the precipitating reagent or reagents, moieties such as bacterial components as well as viral entities, together with cellular constituents such as leukocytes, erythrocytes, epithelial cells and the like, can be selectively removed from the mixture. This feature obviously has benefits, especially in the preparation of injectables, as well as in the preparation of food products, which are prone to microbial degradation or alteration.

In a further exemplary feature, biological proteinaceous mixtures, such as plasma, usually contain components possessing enzymatic or proteolytic activity, such as proteases, plasmin, and the like, which tend to degrade the proteinaceous substrates, thereby altering the composition of the mixture during storage. The present invention is uniquely suited for isolating desired constituents from such components possessing undesired proteolytic activity alleviating the present problems associated with storing such biological entities. The types of biological protein populations that can be isolated in accordance with this invention include, for example, gamma-globulins, albumin, fibrinogen, transferrin, lipoproteins, alpha-globulins, Factor VIII, and the like.

Still a further exemplary feature of the present invention resides in providing precipitating reagents that can be easily sterilized by simple filtration prior to use and can be completely removed from the protein isolates if desired by simple available methods, such as ultrafiltration, electrodialysis, dialysis, or the like. In addition, the precipitating reagents are remarkably non-toxic, compatible with human biochemistry and easily assimiliable even by parental routes of administration and most certainly by peroral administration as in the case of food products. Furthermore, the precipitating reagents of this invention are readily available and inexpensive. With respect to laboratory equipment, any suitable equipment to facilitate collection of protein isolates that is readily available can be used with the methods of this invention. If desired, the entire procedure can be accomplished in a totally closed system. Further, the procedure advantageously lends itself to large scale production and automation.

In still a further exemplary feature, delicate protein populations, such as IgM and IgA fractions, which are primarily responsible for agglutinating activity in blood typing sera and many other serologic procedures, remain intact and do not suffer the denaturation and decomposition inflicted by other isolation methods heretofore available. Thus, the amazing gentleness of the methods of this invention is demonstrated since blood group antibodies are among the most labile with respect to environmental changes, such as, chemical treatment, mechanical forces and the like. It is therefore reasonable to assume that, due to the precision of the methods of this invention, together with their gentle handling of fragile, highly labile constituents, it may be possible to isolate and concentrate other desirable entities such as properdin, which offer great promise therapeutically and which cannot presently be prepared in large volumes with extant technology.

Because of the precision and reproducibility associated with the methods of the present invention, precise fractions and subfractions can be consistently and advantageously prepared time after time which are representative of desired protein populations. In addition, the speed of preparation utilizing the methods of this invention is remarkable. Individual isolates are formed generally immediately upon the addition of the precipitating reagents and the speed of preparation of the final isolate is limited usually only by separation methods employed which may include simple filtration or centrifugation.

Thus, it can be appreciated that the special features and unique advantages of the methods and precipitating reagents of this invention make the same highly effective for isolating desired or specific protein populations from proteinaceous materials in high yields which are highly concentrated, substantially pure and substantially biologically active.

The above and other features and advantages of the invention, including various novel details of the methods and types of precipitating reagents used therewith, will now be more particularly described with reference in the detailed description and pointed out in the examples and claims. It should be understood that the methods and precipitating reagents for isolating proteins embodying the invention are shown in the examples by way of illustration only and not as a limitation of the invention. The principles and feature of this invention may be employed in various and numerous embodiments without departing from the scope of the invention.

### DETAILED DESCRIPTION

By way of illustrating and providing a better appreciation of the present invention, the following detailed description and examples are given concerning methods of this invention for isolating proteins and precipitating reagents utilized therewith and their properties and characteristics.

In accordance with the present invention, it is directed to providing a high resolution method for selectively and sequentially isolating in high yields a specific protein population from a proteinaceous material based upon the physical and chemical characteristics inherent in, and/or peculiar to, the specific protein population. This is accomplished in the present instance by providing a critical concentration of a precipitating reagent comprised of an alkali metal salt of a polyfunctional organic acid which corresponds to a specific protein population to be isolated, adding the precipitating reagent at a concentration less than about the critical concentration to the proteinaceous material for isolating therefrom an undesired protein population, and adding the precipitating reagent at essentially the critical concentration to the proteinaceous material to substantially isolate therefrom the desired protein population. Thus, a specific concentration can be selectively and sequentially isolated in precipitate form from a proteinaceous material containing both the desired as well as undesired protein populations. This can also be accomplished with slight modification by simply reversing the order in which the critical concentration of the precipitating reagent is added to the proteinaceous material. For instance, the precipitating reagent can be added at essentially the critical concentration to a proteinaceous material to isolate therefrom a gross protein population which contains a desired protein population, collecting and resolubilizing the gross protein population in an aqueous liquid, and adding the precipitating reagent at a concentration less than about the critical concentration to the resolubilized gross protein population for substantially isolating in the liquid in soluble form the desired protein population. Thus, by simply modifying the technique, the undesired, rather than desired, protein population is sequentially removed by precipitation from the solubilizing liquid to substantially isolate therein the desired protein population. With respect to resolubilizing the gross protein population, it can be resolubilized in any suitable aqueous liquid such as water, normal saline or mixtures thereof. Regardless of which technique is selected to isolate a desired protein population, the precipitating reagent preferably contains a buffer for controlling and adjusting pH.

In the specification, the term “critical concentration” refers to a specific concentration which consistently selects for a specific protein population of a proteinaceous material. In other words, a specific concentration of a precipitating reagent of this invention is uniquely characteristic for a specific protein population containing the same protein constituents time after time.

In a preferred embodiment, a precipitating reagent of this invention comprises an alkali metal salt of a polyfunctional organic acid and a buffer. Compounds derived from citric acid and tartric acid which are included in this broad class of polyfunctional organic acids are preferably employed, and especially those compounds derived from citric acid. With respect to the alkali metal ions associated with the polyfunctional organic acids, preferably they should be of a monovalent nature such as lithium, potassium and sodium. Therefore, lithium citrate, potassium citrate and sodium citrate are preferred compounds and potassium and sodium citrates are especially preferred compounds to be used in formulating the precipitating reagents of this invention. The most preferred compounds are tri-potassium citrate monohydrate and tri-sodium citrate dihydrate. The precipitating reagents of this invention in selected concentrations are astonishingly and unexpectedly characteristic for specific protein populations in proteinaceous materials.

In addition to being highly specific as aforesaid, the precipitating reagents are remarkably gentle throughout the isolating procedures. Because these precipitating reagents are uniquely specific and surprisingly gentle, the specific or desired protein populations isolated in accordance with this invention are highly concentrated, substantially pure and substantially biologically active. By the term “substantially pure”, it is meant that at least about 90% and usually at least about 95% of a theoretical total of specific protein population is isolated from a proteinaceous material. By “substantially biologically active”, it means that no appreciable losses of specific activity are deleted with a specific protein population isolated, concentrated and recovered. In other words, when specific protein populations are isolated with the methods and reagents of this invention, they generally remain intact and experience little, if any, denaturation and decomposition encountered with the isolation methods heretofore available. Thus, the present invention uniquely provides novel methods and precipitating reagents for isolating specific protein populations in high yields that are highly concentrated, essentially pure and that retain practically all of their biological activities.

In formulating a precipitating reagent for use with the methods of this invention, it is found that the precipitating reagents when derived from, for instance, and alkali metal salt of citric acid, can be prepared in various concentrations without substantially altering the results achieved. It should be understood, however, that the final concentration of citrate in the formulated reagent should be in an effective amount such that when it is added to a proteinaceous material it can reproducibly isolate a selected protein population from a proteinaceous material. To this end, it is found that saturated aqueous solutions of tri-potassium citrate monohydrate and tri-sodium citrate dihydrate are especially suited for accomplishing such results, particularly when the proteinaceous material is plasma. A preferred precipitating reagent containing a saturated solution of tri-sodium citrate dihydrate comprises about a 1.53 molar solution of tri-sodium citrate dihydrate adjusted to a pH of approximately 7 with citric acid, which is approximately 450 milligrams per ml of citrate salt, whereas a preferred precipitating reagent of tri-potassium citrate monohydrate comprises approximately 800 milligrams per ml of citrate salt having a pH adjusted to approximately 7 with hydrochloric acid. Alternately, precipitating reagents containing solutions of tri-potassium citrate monohydrate and tri-sodium citrate dihydrate in concentrations less than saturated concentrations may also be utilized. In addition, precipitating reagents may contain concentrations of only tri-potassium citrate monohydrate or tri-sodium citrate dihydrate or any suitable combination thereof.

Upon adding a precipitating reagent at a critical concentration to a proteinaceous material under one method of this invention, a specific protein population in the nature of a precipitate forms which can be easily isolated, for instance, by centrifugation, filtration, or a combination thereof, or any other suitable technique well known in the art. The filtrate generally contains, quantitatively, the added citrate buffer, indicating that no chemical reaction occurs with the protein of the proteinaceous material which is being precipitated. The addition of a second higher (critical) concentration aliquot of a precipitating reagent to the filtrate of fraction 1 results in the formation of a second precipitate; and the addition of still another higher (critical) concentration aliquot to the filtrate of fraction 2 results in the formation of another precipitate, and so on. Therefore, because of the unique feature that a specific amount of precipitating reagent is characteristic for a specific protein population within a proteinaceous material, an astonishingly large number of different protein fractions can be selectively and sequentially obtained with this invention.

To better illustrate this point, all protein populations from gamma globulin through pre-albumin which are derived from plasma can be collected by adjusting the total citrate content of the plasma to about 600 milligrams per ml. If preferred, two fractions can be obtained by first adjusting to 300 milligrams per ml followed by adjustment of the remainder to 600 milligrams per ml. Or, if desired, it is theoretically, if not actually, possible to obtain, for example, 6,000 separate fractions of plasma proteins by increasing the citrate concentration in the plasma from 40 through 600 milligrams per ml in 0.1 milligram per ml increments. If, however, some protein entities are present in only microgram quantities, they possibly will not give visible precipitates and will go undetected. Therefore, at this point, the detection of protein populations in accordance with the teachings of this invention is possible limited by the lack of precise, sensitive, analytical tools with which to fully evaluate the many possible subfractions. Thus, the high resolution methods of the present invention are extremely unique in that they provide novel means for preparing a myriad of desired protein populations from proteinaceous materials in high yields with a degree of concentration, purity and biological activity that has not been heretofore achieved. Of course, if the alternative method is employed, i.e., to isolate the desired protein population in solubilized form in the resolubilizing liquid, the precipitating reagent preferably should be removed by any suitable know technique, such as reverse osmosis, dialysis or the like.

When fractionating proteins from plasma, it is preferred to utilize a precipitating reagent containing a saturated solution of buffered tri-sodium citrate dihydrate. For instance, it is found that such a reagent ideally selects for protein populations that exist in the plasma which correspond to plasma citrate concentrations ranging from about 40 milligrams per ml up to about 355 milligrams per ml. Presently, no measurable amounts of plasma proteins are detected within the plasma below plasma citrate concentrations of about 40 milligrams per ml. This is not to say, however, that protein populations do not exist in the plasma at plasma citrate concentration less than about 40 milligrams per ml. For protein populations which are isolated within the plasma at plasma citrate concentrations in excess of about 355 milligrams per ml, these plasma proteins can be easily isolated by adjusting the plasma citrate concentration with an additional precipitating reagent containing a saturated solution of tri-potassium citrate monohydrate up to about 700 mg/ml of tri-potassium citrate. The citrate reagents are added preferably in a stepwise fashion in increasing concentrations, each addition of which engenders a different protein population in the precipitate formed thereby. It should be appreciated that all the reagents utilized in accordance with the practices of this invention are readily available and generally inexpensive. Further, the alkali metal salts of polyfunctional organic acids may be prepared, if desired, in accordance with any know method so long as the compounds produced from those methods are suitable for use with this invention.

In some instance, the precipitate formed may contain other undesirable materials. In these cases, it may be necessary to remove such contaminants by processes such as ultrafiltration or electrodialysis.

A convenient means to control and regulate the pH of a precipitating reagent is by the use of a buffer. Any suitable buffer may be utilized to formulate and adjust the pH of the precipitating reagents of this invention. In particular, it is found that hydrochloric acid and citric acid generally achieve the better results. Of course, it should be appreciated that the pH of the precipitating reagents will vary depending upon the proteinaceous materials and the specific protein populations to be isolated therefrom. In operation, the entire process can be generally conducted at a pH ranging from about 4 through about 9. However, when working with human proteins, a pH range from about 7 to about 7.55 and preferably from about 7.35 to about 7.45 should be employed to avoid adversely affecting the specific protein populations being isolated. For instance, deviation from these ranges may result in the denaturing of the tertiary and quaternary structures directly resulting in loss of desirable biological activity. Further, exposure to extremely acidic or alkalinic conditions can result in the alteration of even secondary protein structures by, for example, hydrolysis of disulfide linkages, deamination, etc., which results in a basically denatured, oftentimes aggregated or cross-linked product possessing few, if any, of the desirable characteristics of the protein entities as found in vivo.

In practicing the methods of this invention, they can be easily adapted to isolate large volumes of specific protein populations from various proteinaceous materials which would have practical application in the areas of, for instance, both therapeutics and diagnostics. For example, to a pool of normal plasma, the total protein content can be adjusted to about 5 with normal saline, sterilely filtered by known techniques and held and processed at about 2 to about 10° C. (This filtration removes precipitated fibrin particles, bacteria, red blood cells, etc.). Plasma containing a high amount of free lipid can be defatted using freon if desired; however, free lipid can be removed with relative ease in the early stages of fractionation by a second filtration of chilled supernatent through a 0.22 micron filter, which retains substantially all free lipid (cryo-aggregated chylomicra). A continuous-flow centrifuge (cup type), automatic citrate analyzer (flow cell, ultraviolet, autoanalyzer, wet chemistry system, etc.) coupled with a computer trigger gate to control automatic addition of sufficient citrate solution to attain the desired concentration and, at once, calculate the total volume in the system, enables large volume processing. The entire isolation flow chart can be easily automated and preformed in a completely closed, sterile, aseptic system. A battery of series-connected units, as described above, would enable multiple collection of fractions and subfractions as desired, in a continuous operation necessitating only removal of precipitate from the centrifuge cup as it is collected. The entire procedure, prior to batch processing, can be worked out in the laboratory rendering precise predictability of batch results utilizing relatively simple equipment. (For instance, 10 rough fractions might be prepared in the laboratory and analyzed for desired constituents. The same exact concentration of citrate would be employed in the batch process as in the laboratory.)

Following the initial filtration step, isolating can proceed and probably will best be accomplished by first taking about five or six gross fractions so as to eliminate fibrinogen and albumin from the system. The removal of these two results in a more predictable pattern of subfractionation, since they both, particularly albumin, contribute to the overall ionic strength of the supernatant; and since they are both in relatively high concentration they should be removed from the system prior to the adjustment of ionic strength with buffer. Additionally, it is noted that “filtration” must be of a very gentle nature to obviate or at least minimize shearing of proteins which causes denaturation. For this reason, gentle centrifugation is preferred in separating precipitates (or flotates) from mixtures of continuous and interrupted phases.

The final supernatent may be filtered by reverse osmosis, cleaned up by dialysis or some other large batch process method which can retain molecular weight species of about 1,000 or more, but passes the balance of the salts, etc. The precipitating reagent which comprises a citrate can also be recovered by this method if desired. It should be appreciated that approximately 100% of the mixed salt solution is recoverable in an unchanged form.

In addition to preparing relatively large amounts of protein fractions from plasma for use in vitro diagnostics and in vivo therapeutics, the methods of this invention can well be utilized in the diagnostic laboratory so as to greatly enhance, for example, the sensitivity of any methodology of dealing with the detection of immunoglobin levels such a R.I.A., crossmatching whole blood, tissue typing, HIV testing, and other antibody screening procedures.

The advantages of the methods and precipitating reagents of this invention are ideally suited for isolating a variety of highly useful protein populations from various proteinaceous materials. Exemplary types of proteinaceous materials that can be utilized as sources for protein populations include animal or vegetable fluids and extracts, such as blood or plasma as mentioned above, urine, amniotic or fetal or placental fluid, milk, liver extract, animal feces, corn and soybean extract, as well as other suitable protein sources natural, synthetic or otherwise.

As aforesaid, protein populations isolated according to the teachings of this invention are high concentrated, generally non-aggregated, substantially biologically active, substantially pure and in some cases suitable for intravenous injection. Suggested applications which might be possible for such protein populations isolated in accordance with this invention may include employment in a variety of applications, such as preventative and therapeutic. For instance, hyperimmune plasma products can be utilized intravenously enabling optimum concentration of desired constituents to be obtained in vivo. Further, specific immune serums against bacteria, viruses and the like occurring in both human beings and animals possibly can be prepared, concentrated and injected intravenously to establish more predictable levels of therapeutic immunoglobins, such as anti-staphylococcus, anti-hepatitis, anti-pseudomonas, etc., especially valuable in light of the ubiquitous emergence of antibiotically-tolerant strains of pathogenic organisms. With respect to viral isolations, they can be utilized for viral characterization and study as well as preparation of attenuated strains for utilization in immunogen production for preparations of immunoglobulins. Likewise, passively-transferred anti-human spermatozoa flagellar antibodies could be produced in non-fertile females, isolated, concentrated and injected into fertile females as an alternative method birth control. Still further, specific protein subfractions utilized for therapy and prevention of diseases can be isolated and concentrated from normal plasma. For example, transfer factor, properdin and interferon could be collected to prevent replication of RNA in tumor cells, myelomas, and leukemias. Similarly, transferrin and ceruloplasmin could be isolated and utilized in the control of hemochromatosis and Wilson's disease, respectively. Betacryoglobin (Factor VIII) can be collected in high levels in intact form by the teachings of this invention which advantageously eliminate the freezing process heretofore required with other methods. In addition, it may be possible to prepare typing serum, RhoGam, Factor VIII, etc. from single units of plasma. Still further, the isolation methods could be applicable to urine and feces as means for isolating and concentrating natural as well as other occurring proteins including those observed in pathologic and altered states for diagnostic purposes. Also, proteins and peptides, such as hormone constituents, of urine possibly can be collected or harvested for purposes of generating therefrom therapeutic and preventative substances. Still further, the method possibly could be utilized in the performance of routine diagnostic and preparative medical testing protocols in order to increase the sensitivities of such protocols by virtue of the concentration of the desired constituents. For instance, tissue typing, viral isolation, pretransfusion cross-matching, detecting low levels of antibodies such as those associated with viral pathologies like hepatitis, HIV, mononucleosis, common cold, HIV and the like. With respect to proteinaceous materials derived from dairy products, it is possible to remove casein quickly and in a highly pure state, as well as the other milk products, which could possibly result in a complete separation process for milk with no whey, as it is known today, as a by-product. Further, by-product fluids from dairy fermentation processes could be isolated for the recovery of microbial enzymes, etc. It should be understood, however, that the above-suggested applications are exemplary of possible uses of specific protein populations isolated with the methods of this invention, and that such uses may be limited, for example, by a suitable method of detection of any given protein population that is isolated herewith.

To identify the protein populations isolated in accordance with the teachings of this invention, flourescein isothiocyanate, rhodamine-B isothiocyanate, and the like, can be used to tag the proteins contained in each fraction unhindered by albumin which usually plagues similar analytical techniques by causing high background noise in the detector unit. Each tagged sample can be analyzed utilizing high pressure liquid chromatography equipped with a fluorescent detector unit. Elution of peaks should be performed in identification of each fraction constituent performed by any suitable method that is available (I.E.P.A., A.I.P., etc.).

The present invention may, of course, by carried out in other specific ways than those herein set forth without departing from the spirit and essential characteristics of the invention. The present embodiments are, therefore, to be considered in all respects as illustrative and not restrictive, and any changes coming within the meaning and equivalency range of the appended claims are to be embraced therein.

What is claimed is:

1. A high resolution method of isolating a desired protein population from a proteinaceous material comprising:
providing a critical concentration of a precipitating reagent comprised of an alkali metal salt of a polyfunctional organic acid which corresponds to the desired protein population to be isolated;
adding the precipitating reagent at a concentration less than about the critical concentration to the proteinaceous material for isolating therefrom an undesired protein population; and
adding the precipitating reagent at essentially the critical concentration to the fractionated proteinaceous material to further isolate therefrom the desired protein population wherein said desired protein population is concentrated, substantially pure and biologically active.

2. The method of claim 1 wherein the desired protein population is selected from the class consisting of an animal protein or vegetable protein.

3. The method of claim 2 wherein the animal protein is selected from the class consisting of blood protein, plasma protein, urine protein, milk protein, feces protein or amniotic or placental protein.

4. The method of claim 1 wherein the proteinaceous material is selected from the class consisting of animal fluid, animal extract, vegetable fluid or vegetable extract.

5. The method of claim 4 wherein the animal fluid is selected from the class consisting of blood, plasma, urine, milk or amniotic fluid.

6. The method of claim 4 wherein the animal extract is selected from the class consisting of liver extract or animal feces.

7. The method of claim 1 wherein the desired protein population is a virus or protein particles derived from the virus.

8. The method of claim 1 wherein the polyfunctional organic acid selected from the class consisting of citric acid or tartric acid.

9. The method of claim 1 wherein the alkali metal of a polyfunctional organic acid is selected from the class consisting of lithium citrate, potassium citrate, sodium citrate.

10. The method of claim 1 wherein the alkali metal of a polyfunctional organic acid is selected from the class consisting of tri-potassium citrate monohydrate or tri-sodium citrate dihydrate.

11. The method of claim 1 wherein the precipitating reagent further comprises a buffer.

12. The method of claim 11 wherein the buffer is selected from the class consisting of citric acid or hydrochloric acid.

13. The method of claim 11 wherein the precipitating reagent comprises tri-sodium citrate dihydrate and a suitable amount of citric acid for adjusting pH.

14. The method of claim 11 wherein the precipitating reagent comprises tri-potassium citrate monohydrates and a suitable amount of hydrochloric acid for adjusting pH.

15. The method of claim 11 wherein the precipitating reagent is adjusted to a pH ranging from about 4 to about 9.

16. A high resolution precipitating reagent for selectively isolating a desired protein population from a proteinaceous material comprising:
an alkali metal of a polyfunctional organic acid in a concentration suitable for selectively isolating the desired protein population; and
a buffer for adjusting pH of said reagent whereby the desired protein population is selectively isolated from the proteinaceous material in a concentrated, substantially pure and biologically active form.

17. A precipitating reagent of claim 16 wherein said alkali metal of a polyfunctional organic acid is selected from the class consisting of lithium citrate, potassium citrate, sodium citrate or mixtures thereof.

18. The precipitating reagent of claim 17 wherein potassium citrate is tri-potassium citrate monohydrate.

19. The precipitating reagent of claim 18 wherein said tri-potassium citrate monohydrate is in a concentration ranging from about 10 to about 700 milligrams per ml of citrate.

20. The precipitating reagent of claim 17 wherein said sodium citrate is tri-sodium citrate dihydrate.

21. The precipitating reagent of claim 20 wherein said tri-sodium citrate is in a concentration ranging from about 30 to about 350 milligrams per ml of citrate.

22. The precipitating reagent of claim 16 wherein buffering agent is selected from the class consisting of citric acid or hydrochloric acid.

23. The precipitating reagent of claim 16 wherein said reagent has a pH ranging from about 4 to about 9.

24. The precipitating reagent of claim 18 wherein said buffering agent is hydrochloric acid.

25. The precipitating reagent of claim 20 wherein said buffering agent is citric acid.

26. A high resolution method of isolating a desired protein population from a proteinaceous material comprising:
providing a critical concentration of a precipitating reagent comprised of an alkali metal of a polyfunctional organic acid which corresponds to the desired protein population to be isolated;
adding the precipitating reagent at essentially the critical concentration to the proteinaceous material to precipitate therefrom a gross protein population containing the desired protein population in an aqueous liquid; and
adding the precipitating reagent at a concentration less than about the critical concentration to the resolubilized gross protein population for further precipitating therefrom an undesired protein population to substantially isolate in the liquid in soluble form the desired protein population.

27. The method of claim 26 wherein the desired protein population is selected from the class consisting of an animal protein or vegetable protein.

28. The method of claim 27 wherein the animal protein is selected from the class consisting of blood protein, plasma protein, urine protein, feces protein or amniotic and placental protein.

29. The method of claim 26 wherein the proteinaceous material is selected from the class consisting of animal fluid, animal extract, vegetable fluid or vegetable extract.

30. The method of claim 29 wherein the animal fluid is selected from the class consisting of blood, plasma, urine, milk or amniotic or placental fluid.

31. The method of claim 29 wherein the animal extract is selected from the class consisting of liver extract or animal feces.

32. The method of claim 26 wherein the desired protein population is a virus or protein particles derived from the virus.

33. The method of claim 26 wherein the polyfunctional organic acid is selected from the class consisting of lithium citrate, potassium citrate, or sodium citrate.

34. The method of claim 26 wherein the alkali metal salt of a polyfunctional organic acid is selected from the class consisting of lithium citrate, potassium citrate, or sodium citrate.

35. The method of claim 26 wherein the alkali metal salt of a polyfunctional organic acid is selected from the class consisting of tri-potassium citrate monohydrate or tri-sodium citrate dihydrate.

36. The method of claim 26 wherein the precipitating reagent further comprises a buffer.

37. The method of claim 36 wherein the buffering agent is selected from the class consisting of citric acid or hydrochloric acid.

38. The method of claim 36 wherein the precipitating reagent has a pH adjusted from about 4 to about 9.

39. The method of claim 26 wherein the aqueous liquid is selected from the class consisting of water, normal saline or mixtures thereof.

40. The method of claim 26 wherein the precipitating reagent comprises tri-sodium citrate dihydrate and a suitable amount of citric acid for adjust pH.

41. The method of claim 26 wherein the precipitating reagent comprises tri-potassium citrate monohydrate and a suitable amount of hydrochloric acid for adjusting pH.
